(19) United States
(12) Patent Application Publication
(10) Pub. No.: US 2019/0209708 A1
(43) Pub. Date: Jul. 11, 2019

(54) NOVEL THERAPEUTIC TOOLS AND METHODS FOR TREATING BLINDNESS

(71) Applicant: FRIEDRICH MIESCHER INSTITUTE FOR BIOMEDICAL RESEARCH, Basel (CH)

(72) Inventors: Botond ROSKA, Oberwil (CH); Santiago ROMPANI, Basel (CH)

(21) Appl. No.: 16/302,222

(22) PCT Filed: May 15, 2017
(86) PCT No.: PCT/IB2017/052851

§ 371 Date: Nov. 16, 2018

(30) Foreign Application Priority Data
May 17, 2016 (EP) 16169912.9

(51) Int. Cl. A61K 48/00; A61P 27/02

(52) U.S. Cl. CPC A61K 48/0058; A61P 27/02; A61K 48/0075

(57) ABSTRACT

The present inventions relates to tools and methods for the treatment of blindness in a patient, wherein a vector comprising a gene coding for a light-sensitive molecule is injected into the lateral geniculate nucleus of the patient.

### FIELD OF THE INVENTION

The present invention relates to methods of treating blindness. The present invention also relates to constructs for use in treating blindness.

### BACKGROUND OF THE INVENTION

Blindness is a major health problem that disables millions of people worldwide. One of the most common cause of blindness is the disfunction of the retina. The three most common forms of retinal blindness are retinitis pigmentosa (RP), macular degeneration (MD) and glaucoma (G). In RP and MD the primary problem is the degeneration of photoreceptors and the consequent loss of photosensitivity. There is thus a need to be able to obviate the problems associated with such degeneration of photoreceptors.

One approach has been to develop a retinal prosthesis, a “seeing eye” chip with as many as 1,000 tiny electrodes to be implanted in the eye. This would have the potential to help people who have lost their sight to regain enough vision to function independently, but the numbers of electrodes is simply insufficient to provide a high degree or level of sight to be obtained. Moreover, there are problems associated with inserting foreign bodies into the eye.

Recently a number of genes has been isolated and/or manipulated that when expressed can make cells light sensitive. In some cases additional non-genetic factors are also needed to make cells light sensitive.

One proposal by Eli in 2001 was to use the chlorophyll-containing proteins in spinach to treat vision loss. These proteins give off a small electrical voltage after capturing the energy of incoming photons of light. Although, the research has shown that photosystem I reaction centres can be incorporated into a liposome and are shown to be functional, in that it produces the experimental equivalent of a voltage when light is shone on it, hitherto this has not been shown to work in a retinal cell.

Other work by neurobiologist Richard Kramer at UC Berkeley has looked at re-engineering a potassium channel to be responsive to light rather than voltage, in order to allow insertion of a light activated switch into brain cells normally insensitive to light. However, the channel has to be mutated so that it always stays open and a chemical “plug”, attached to the channel, which is sensitive to light such that when lit with long-wavelength UV light, the plug is released from the channel, letting potassium out of the channel. Light of a longer wavelength causes the plug to insert back into the channel and stop release of potassium. It will be appreciated however, that such a system is extremely complex and problems are likely to arise if the channel is delivered to the wrong type of retinal cells.

Bi et al., (Neuron, 50, 2006, p 23-33) discloses the use of microbial-type rhodopsin to restore visual responses in mice with photoreceptor degeneration. However, the expression of the rhodopsin gene is likely to have occurred in a variety of types of cell in the eye which is potentially undesirable and/or problematic. It also appears that the threshold light intensity required for producing responses is much higher than for normal rod and cone photoreceptors, but there is no teaching of how this may be addressed in, for example, low light environments.

An alternative methods have been described by some of the present inventors, e.g. in WO-A-2008/022772.

However, in some eye diseases such as glaucoma, late stage diabetic retinopathy, hereditary optic neuropathies as well as optic nerve injuries the output cells of the retina, the ganglion cells, degenerate, such that strategies that seek to restore vision in patients suffering from these diseases cannot rely on a repair in the retina. The inventors therefore considered a strategy for vision restoration in these patients by activating cells downstream of the retina.

### SUMMARY OF THE INVENTION

The visual signal is initially processed in the retina and most of conscious vision is then relayed to the lateral geniculate nucleus (LGN) of the thalamus, which in turn projects to the primary visual cortex. Since the visual signal is processed less in the LGN than in the cortex, and the number of cells dedicated to the same visual angle or retinal area is smaller in the LGN than in the cortex, the present inventors considered a strategy involving stimulating the cells of the LGN in order to restore vision. However, the small size of the LGN, which is found deep within the brain, renders the stimulation of discrete parts thereof difficult. The inventors therefore had the idea to optogenetically activated LGN cells using a vector comprising a gene coding for a light-sensitive molecule and to illuminate with visual patters the axon terminals of LGN cells where they form connections with the visual cortex, in the more accessible surface of the brain. To address the question of whether LGN cell stimulation could evoke meaningful responses in blind or normal-sighted animal, the inventors infected either normal or blind mice with channelrhodopsin in the LGN via an AAV, as well as with GCAMP in the cortex. This allowed them to stimulate the axon terminals of LGN cell in the cortex and observe if responses could be evoked in cortex. In these experiments the inventors found that channelrhodopsin expressed in the LGN cell axon terminals of mice could activate cells in acute slices of the visual cortex, where the LGN axon terminals were cut off from their cell bodies. The inventors also found that V1 cells in vivo responded to such stimulation as well. This finding suggests that optogenetic stimulation of LGN axons is sufficient to evoke responses in visual cortex. The present invention thus encompasses a method for the treatment of blindness in a patient, wherein a vector comprising a gene coding for a light-sensitive molecule is injected into the lateral geniculate nucleus of the patient. In some embodiments, the method of the invention further comprising the step of exposing the visual cortex of said patient to light signals. Suitable light-sensitive molecules are halorhodopsins and channelrhodopsins. In some embodiments, different vectors are injected at different location of the lateral geniculate nucleus of the patient, thus leading to the expression of different exogenous genes by different populations of LGN cells. In a preferred embodiment, at least two different vectors carrying genes coding for at least two different light-sensitive molecules are used, so that different populations of LGN cells will react to light of different wave lengths. In some embodiments, combinations of channelrhodopsins and halorhodopsins are used. Suitable promoters for the methods of the invention are the Human elongation factor-1 alpha (EF-1 alpha), the Human cytomegalovirus promoter (CMV) and the CAG promoter.

The present invention also encompasses a vector comprising a gene coding for a light-sensitive molecule for use in a method of treating blindness by injection into the LGN. Suitable light-sensitive molecules are halorhodopsins and channelrhodopsins. The expression of the light-sensitive molecule can be controlled by a promoter selected from the group of Human elongation factor-1 alpha (EF-1 alpha), Human cytomegalovirus promoter (CMV) or CAG promoter.

The present invention further encompasses a kit comprising at least two different vectors comprising genes coding for light-sensitive molecules for use in a method of treating blindness by injection into the LGN.

### DESCRIPTION OF THE FIGURE

FIG. 1: Trace showing in primary visual cortex lack of response in one cell (bottom trace) and a cell showing chr2-mediated light response (top trace).

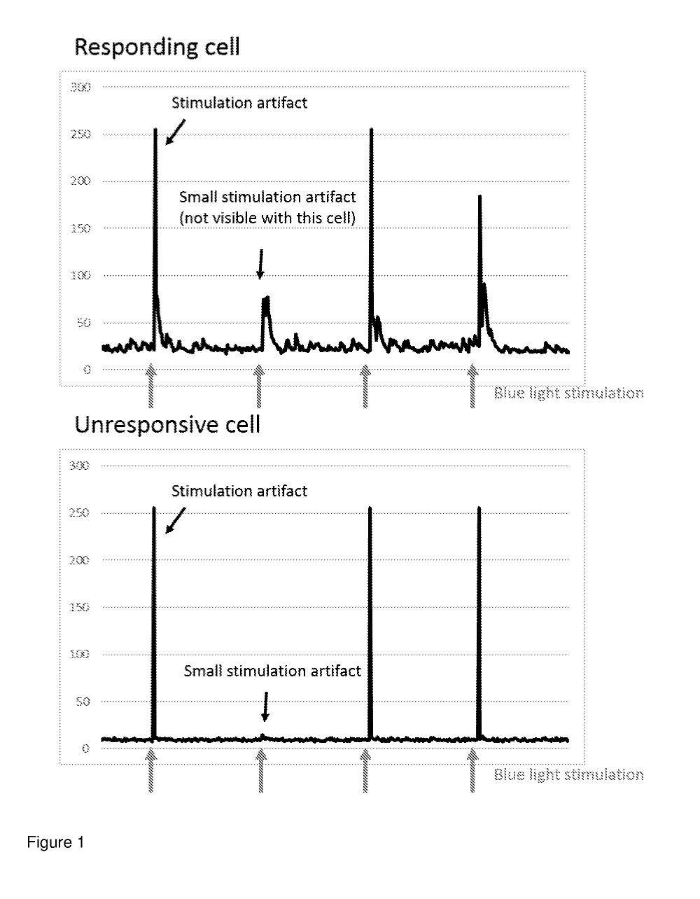

### DETAILED DESCRIPTION OF THE INVENTION

The visual signal is initially processed in the retina and most of conscious vision is then relayed to the lateral geniculate nucleus (LGN) of the thalamus, which in turn projects to the primary visual cortex. Since the visual signal is processed less in the LGN than in the cortex, and the number of cells dedicated to the same visual angle or retinal area is smaller in the LGN than in the cortex, the present inventors considered a strategy involving stimulating the cells of the LGN in order to restore vision. However, the small size of the LGN, which is found deep within the brain, renders the stimulation of discrete parts thereof difficult. The inventors therefore had the idea to optogenetically activated LGN cells using a vector comprising a gene coding for a light-sensitive molecule and to illuminate with visual patters the axon terminals of LGN cells where they form connections with the visual cortex, in the more accessible surface of the brain. To address the question of whether LGN cell stimulation could evoke meaningful responses in blind or normal-sighted animal, the inventors infected either normal or blind mice with channelrhodopsin in the LGN via an AAV, as well as with GCAMP in the cortex. This allowed them to stimulate the axon terminals of LGN cell in the cortex and observe if responses could be evoked in cortex. In these experiments the inventors found that channelrhodopsin expressed in the LGN cell axon terminals of mice could activate cells in acute slices of the visual cortex, where the LGN axon terminals were cut off from their cell bodies. The inventors also found that V1 cells in vivo responded to such stimulation as well. This finding suggests that optogenetic stimulation of LGN axons is sufficient to evoke responses in visual cortex. The present invention thus encompasses a method for the treatment of blindness in a patient, wherein a vector comprising a gene coding for a light-sensitive molecule is injected into the lateral geniculate nucleus of the patient. In some embodiments, the method of the invention further comprising the step of exposing the visual cortex of said patient to light signals. Suitable light-sensitive molecules are halorhodopsins and channelrhodopsins. In some embodiments, different vectors are injected at different location of the lateral geniculate nucleus of the patient, thus leading to the expression of different exogenous genes by different populations of LGN cells. In a preferred embodiment, at least two different vectors carrying genes coding for at least two different light-sensitive molecules are used, so that different populations of LGN cells will react to light of different wave lengths. In some embodiments, combinations of channelrhodopsins and halorhodopsins are used. Suitable promoters for the methods of the invention are the Human elongation factor-1 alpha (EF-1 alpha), the Human cytomegalovirus promoter (CMV) and the CAG promoter.

The present invention also encompasses a vector comprising a gene coding for a light-sensitive molecule for use in a method of treating blindness by injection into the LGN. Suitable light-sensitive molecules are halorhodopsins and channelrhodopsins. The expression of the light-sensitive molecule can be controlled by a promoter selected from the group of Human elongation factor-1 alpha (EF-1 alpha), Human cytomegalovirus promoter (CMV) or CAG promoter.

The present invention further encompasses a kit comprising at least two different vectors comprising genes coding for light-sensitive molecules for use in a method of treating blindness by injection into the LGN.

Compositions comprising the nucleic acid molecules of the invention are also encompassed by the present invention. Said compositions can be pharmaceutically acceptable compositions.

“Retinal photoreceptors” comprise rods and cones. The retina can be viewed as a parallel image processor that acquires images via a mosaic of photoreceptors and that extracts various visual features from the acquired images. Rod photoreceptors respond directly to light at lower intensities and cone photoreceptors at higher intensities. The cellular infrastructure that underlies parallel processing consists of mosaics of local neuronal circuits. The retina has ˜20 such circuit mosaics, built from more than 60 cell types, which independently extract different features from the visual world. Each mosaic has an associated mosaic of output cells, the ganglion cells, which relay the computed feature to higher brain centers. Each cone in the retina is connected to around 10 types of cone bipolar cells, and each of these bipolar cells is connected to several types of ganglion cells. Cones, bipolar cells, and ganglion cells use the excitatory neurotransmitter glutamate to communicate. Communication between cones and bipolar cells is modified by the inhibitory horizontal cells, and communication between bipolar cells and ganglion cells is modified by a large variety of inhibitory amacrine cells. Cones respond to light by lowering their membrane voltage; i.e., they hyperpolarize. Half of the cone bipolar cells also hyperpolarize (OFF cells), whereas the other half increase their membrane voltage, depolarizing when light intensity increases (ON cells). The polarity of the ganglion cell responses is determined by the polarity of the bipolar cells from which they receive input. Each rod is connected to a special bipolar cell type called the rod bipolar cell. Rod bipolar cells “talk” to the so-called AII amacrine cells, which then provide excitatory input to the axon terminals of ON cone bipolar cells and inhibitory input to OFF cone bipolar cell terminals. Rods (photoreceptors) are hyperpolarized by light, whereas rod bipolar cells and AII amacrine cells are depolarized: These are therefore ON cells. Retinal cells are arranged in mosaics, covering the entire retina. The only exception to the mosaic arrangement is a special area of the retina in some primates and in a few predatory birds and reptiles. This area is called the fovea and is the place with the highest cone density. The human fovea, also called macula, has no rods within its center, and the only cellular compartment that is organized in a mosaic fashion is the cone outer segment. Foveal cone cell bodies are piled on top of each other, whereas cell bodies of all other cell types are shuffled to the side, forming a concentric ring of cell bodies.

By “blindness” is meant total or partial loss of vision. Typically the medicament may be used to treat blindness associated with glaucoma, late stage diabetic retinopathy, hereditary optic neuropathies as well as optic nerve injuries the output cells of the retina, the ganglion cells. However, it is to be appreciated that any disease or condition which leads to degeneration or non-functioning of photoreceptors in the eye may also be treated using the medicament.

An “active fragment of a light-sensitive molecule” is a fragment which when expressed generates a polypeptide which is still capable of functioning as a light capturing molecule which causes a subsequent flow of ions in or out of the cell in which the molecule is located and a consequent change in voltage.

By “hyperpolarisation” is meant the decrease of the membrane potential of a cell (made more negative). By “depolarisation” is meant the increase of the membrane potential of a cell (made more positive).

Many different viral and non-viral vectors and methods of their delivery, for use in gene therapy, are known, such as adenovirus vectors, adeno-associated virus vectors, retrovirus vectors, lentiviral vectors, herpes virus vectors, liposomes, naked DNA administration and the like. A detailed review of possible techniques for transforming genes into desired cells of the eye is taught by Wright (Br J Ophthalmol, 1997; 81: 620-622). Moreover, it may also be possible to use encapsulated cell technology as developed by Neurotech, for example.

It is understood that it is preferable that expression of the light-sensitive molecule of the invention is controlled by way of a cell specific promoter. Thus a cell specific promoter may be used to ensure that the light-sensitive molecule is only expressed in a specific cell type.

Once expressed in an appropriate cell, the depolarizing light-sensitive molecule inserts within the plasma membrane of the cell, rendering the cell photosensitive and able to cause ion transport, cation or anion, in response to light.

These and other aspects of the present invention should be apparent to those skilled in the art, from the teachings herein.

For convenience, the meaning of certain terms and phrases employed in the specification, examples, and appended claims are also provided below.

The singular forms “a,” “an,” and “the” include plural reference unless the context clearly dictates otherwise.

“Halorhodopsin” is a light-driven ion pump, specific for chloride ions, and found in phylogenetically ancient “bacteria” (archaea), known as halobacteria. It is a seven-transmembrane protein of the retinylidene protein family, homologous to the light-driven proton pump bacteriorhodopsin, and similar in tertiary structure (but not primary sequence structure) to vertebrate rhodopsins, the pigments that sense light in the retina. Halorhodopsin also shares sequence similarity to channelrhodopsin, a light-driven ion channel. Halorhodopsin contains the essential light-isomerizable vitamin A derivative all-trans-retinal. Halorhodopsin is one of the few membrane proteins whose crystal structure is known. Halorhodopsin isoforms can be found in multiple species of halobacteria, including H. salinarum, and N. pharaonis (NphR). Much ongoing research is exploring these differences, and using them to parse apart the photocycle and pump properties. After bacteriorhodopsin, halorhodopsin may be the best type I (microbial) opsin studied. Peak absorbance of the halorhodopsin retinal complex is about 570 nm. Recently, halorhodopsin has become a tool in optogenetics. Just as the blue-light activated ion channel channelrhodopsin-2 opens up the ability to activate excitable cells (such as neurons, muscle cells, pancreatic cells, and immune cells) with brief pulses of blue light, halorhodopsin opens up the ability to silence excitable cells with brief pulses of yellow light.

Thus halorhodopsin and channelrhodopsin together enable multiple-color optical activation, silencing, and desynchronization of neural activity, creating a powerful neuroengineering toolbox. Further variants of halorhodopsin have been developed, e.g. enhanced NphR (eNphR). For the purpose of the present invention, said variants are also included in the definition of “Halorhodopsin”.

The light-sensitive molecule can be a light-gated ion channel gene for instance a rhodopsin gene, such as a rhodopsin from a microorganism, such as a unicellular alga, typically from the species Chlamydomonas, especially Chlamydomonas reinhardtii. A preferred rhodopsin is Channelrhodopsin-2 (ChR2) which is a light gated cation channel from C. reinhardtii, see for example, Boyden et al 2005 (Nature Neuroscience, 8, 9; 1263-1268) and WO-A-2003/084994. Variants of Channelrhodopsin-2 are also very suitable for the present invention. An example of such y variant is CatCh, a L132C mutant of ChR2 (Nat Neurosci. 2011 April; 14(4):513-8. doi: 10.1038/nn.2776. Ultra light-sensitive and fast neuronal activation with the Ca²+-permeable channelrhodopsin CatCh. Kleinlogel S1, Feldbauer K, Dempski R E, Fotis H, Wood P G, Bamann C, Bamberg E.).

“Polynucleotide” and “nucleic acid”, used interchangeably herein, refer to polymeric forms of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleotide phosphoramidate or a mixed phosphoramidate-phosphodiester oligomer. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids, PNA and LNA. Polynucleotides may further comprise genomic DNA, cDNA, or DNA-RNA hybrids.

“Sequence Identity” refers to a degree of similarity or complementarity. There may be partial identity or complete identity. A partially complementary sequence is one that at least partially inhibits an identical sequence from hybridizing to a target polynucleotide; it is referred to using the functional term “substantially identical”. The inhibition of hybridization of the completely complementary sequence to the target sequence may be examined using a hybridization assay (Southern or Northern blot, solution hybridization and the like) under conditions of low stringency. A substantially identical sequence or probe will compete for and inhibit the binding (i.e., the hybridization) of a completely identical sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarities (e.g., less than about 30% identity); in the absence of non-specific binding, the probe will not hybridize to the second non-complementary target sequence.

Another way of viewing sequence identity in the context to two nucleic acid or polypeptide sequences includes reference to residues in the two sequences that are the same when aligned for maximum correspondence over a specified region. As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

“Gene” refers to a polynucleotide sequence that comprises control and coding sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene.

“Expression” generally refers to the process by which a polynucleotide sequence undergoes successful transcription and translation such that detectable levels of the amino acid sequence or protein are expressed. In certain contexts herein, expression refers to the production of mRNA. In other contexts, expression refers to the production of protein.

“Cell type” refers to a cell from a given source (e.g., tissue or organ) or a cell in a given state of differentiation, or a cell associated with a given pathology or genetic makeup.

“Polypeptide” and “protein”, used interchangeably herein, refer to a polymeric form of amino acids of any length, which may include translated, untranslated, chemically modified, biochemically modified, and derivatized amino acids. A polypeptide or protein may be naturally occurring, recombinant, or synthetic, or any combination of these. Moreover, a polypeptide or protein may comprise a fragment of a naturally occurring protein or peptide. A polypeptide or protein may be a single molecule or may be a multi-molecular complex. In addition, such polypeptides or proteins may have modified peptide backbones. The terms include fusion proteins, including fusion proteins with a heterologous amino acid sequence, fusions with heterologous and homologous leader sequences, with or without N-terminal methionine residues, immunologically tagged proteins, and the like.

A “fragment of a protein” refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

An “expression product” or “gene product” is a biomolecule, such as a protein or mRNA, which is produced when a gene in an organism is transcribed or translated or post-translationally modified.

“Host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

The term “functional equivalent” is intended to include the “fragments”, “mutants”, “derivatives”, “alleles”, “hybrids”, “variants”, “analogs”, or “chemical derivatives” of the native gene or virus.

“Isolated” refers to a polynucleotide, a polypeptide, an immunoglobulin, a virus or a host cell that is in an environment different from that in which the polynucleotide, the polypeptide, the immunoglobulin, the virus or the host cell naturally occurs.

“Substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated.

“Diagnosis” and “diagnosing” generally includes a determination of a subject's susceptibility to a disease or disorder, a determination as to whether a subject is presently affected by a disease or disorder, a prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived therefrom and the progeny thereof. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components.

“Individual”, “subject”, “host” and “patient”, used interchangeably herein, refer to any mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, and mice.

“Hybridization” refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

“Stringent conditions” refers to conditions under which a probe may hybridize to its target polynucleotide sequence, but to no other sequences. Stringent conditions are sequence-dependent (e. g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e. g., 10 to 50 nucleotides). Stringent conditions may also be achieved with the addition of destabilizing agents, such as formamide.

“Biomolecule” includes polynucleotides and polypeptides.

“Biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

“Oligonucleotide” refers to a polynucleotide sequence comprising, for example, from about 10 nucleotides (nt) to about 1000 nt. Oligonucleotides for use in the invention are for instance from about 15 nt to about 150 nt, for instance from about 150 nt to about 1000 nt in length. The oligonucleotide may be a naturally occurring oligonucleotide or a synthetic oligonucleotide.

“Modified oligonucleotide” and “Modified polynucleotide” refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-5′ linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e. g., hexose phosphodiester; peptide nucleic acids). The modifications may be internal (single or repeated) or at the end (s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e. g., 3′-substituted ribonucleotides or deoxyribonucleotide monomers), any of which are bound together via 5′ to 3′ linkages.

“Biomolecular sequence” or “sequence” refers to all or a portion of a polynucleotide or polypeptide sequence.

The term “detectable” refers to a polynucleotide expression pattern which is detectable via the standard techniques of polymerase chain reaction (PCR), reverse transcriptase- (RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression level it is desired to detect.

A “target protein” refers to a polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e. g., gene or mRNA) whose expression level it is desired to detect.

“Complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

“BLAST” refers to Basic Local Alignment Search Tool, a technique for detecting ungapped sub-sequences that match a given query sequence.

“BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence”, as understood herein, is a group of assembled overlapping sequences, particularly between sequences in one or more of the databases of the invention.

The nucleic acid molecules of the present invention can be produced by a virus harbouring a nucleic acid that encodes the relevant gene sequence. The virus may comprise elements capable of controlling and/or enhancing expression of the nucleic acid. The virus may be a recombinant virus. The recombinant virus may also include other functional elements. For instance, recombinant viruses can be designed such that the viruses will autonomously replicate in the target cell. In this case, elements that induce nucleic acid replication may be required in a recombinant virus. The recombinant virus may also comprise a promoter or regulator or enhancer to control expression of the nucleic acid as required. Tissue specific promoter/enhancer elements may be used to regulate expression of the nucleic acid in specific cell types. The promoter may be constitutive or inducible.

As used herein, the term “promoter” refers to any cis-regulatory elements, including enhancers, silencers, insulators and promoters. A promoter is a region of DNA that is generally located upstream (towards the 5′ region) of the gene that is needed to be transcribed. The promoter permits the proper activation or repression of the gene which it controls. “Specific expression”, also referred to as “expression only in a certain type of cell” means that at least more than 75% of the cells expressing the gene of interest are of the type specified.

Expression cassettes are typically introduced into a vector that facilitates entry of the expression cassette into a host cell and maintenance of the expression cassette in the host cell. Such vectors are commonly used and are well known to those of skill in the art. Numerous such vectors are commercially available, e. g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e. g., LEU2, URA3, TRP 1, HIS3, GFP), centromeric sequences, etc.

Suitable promoters are the human elongation factor-1 alpha (EF-1 alpha; a constitutive promoter of human origin that can be used to drive ectopic gene expression in various in vitro and in vivo contexts), the human cytomegalovirus promoter (CMV) and the CAG promoter, a strong synthetic promoter frequently used to drive high levels of gene expression in mammalian expression vectors.

Viral vectors, for instance an AAV, a PRV or a lentivirus, are suitable to target and deliver genes to LGN neurons.

“Specific expression” means that at least more than 75% of the cells expressing the gene of interest are of the type specified, i.e. LGN neurons in the present case. Examples of promoters which are suitable for the expression of constructs in the present invention are Human elongation factor-1 alpha (EF-1 alpha), Human cytomegalovirus promoter (CMV) or CAG promoter.

Contaminant components of its natural environment are materials that would interfere with the methods and compositions of the invention, and may include enzymes, hormones, and other proteinaceous or nonproteinaceous solutes. Ordinarily, an isolated agent will be prepared by at least one purification step. In one embodiment, the agent is purified to at least about 60%, at least about 65%, at least about 70%, at least about 75%, at least about 80%, at least about 85%, at least about 88%, at least about 90%, at least about 92%, at least about 95%, at least about 97%, at least about 98%, at least about 99%, at least about 99.9%, or at least about 99.99% by weight.

“Expressing” a protein in a cell means to ensure that the protein is present in the cell, e. g., for the purposes of a procedure of interest. In numerous embodiments, “expressing” a protein will comprise introducing a transgene into a cell comprising a polynucleotide encoding the protein, operably linked to a promoter, wherein the promoter is a constitutive promoter, or an inducible promoter where the conditions sufficient for induction are created, as well as a localization sequence. However, a cell that, e. g., naturally expresses a protein of interest, can be used without manipulation and is considered as “expressing” the protein. For the present invention, “specific expression of a depolarizing light-gated ion channel in a retinal photoreceptor” means that the depolarizing light-gated ion channel is expressed exclusively (more than 75%) in a retinal photoreceptor. It is important to note that an expression in many different cells might lead to contradictory signals, annihilating each other.

A “fluorescent probe” refers to any compound with the ability to emit light of a certain wavelength when activated by light of another wavelength.

“Fluorescence” refers to any detectable characteristic of a fluorescent signal, including intensity, spectrum, wavelength, intracellular distribution, etc.

“Detecting” fluorescence refers to assessing the fluorescence of a cell using qualitative or quantitative methods. For instance, the fluorescence is determined using quantitative means, e. g., measuring the fluorescence intensity, spectrum, or intracellular distribution, allowing the statistical comparison of values obtained under different conditions. The level can also be determined using qualitative methods, such as the visual analysis and comparison by a human of multiple samples, e. g., samples detected using a fluorescent microscope or other optical detector (e. g., image analysis system, etc.) An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

An assay performed in a “homogeneous format” means that the assay can be performed in a single container, with no manipulation or purification of any components being required to determine the result of the assay, e. g., a test agent can be added to an assay system and any effects directly measured. Often, such “homogeneous format” assays will comprise at least one component that is “quenched” or otherwise modified in the presence or absence of a test agent.

Methods for expressing heterologous proteins in cells are well known to those of skill in the art, and are described, e. g., in Ausubel (1999), Guthrie and Fink (1991), Sherman, et al. (1982) Methods in Yeast Genetics, Cold Spring Harbor Laboratories, Freshney, and others. Typically, in such embodiments, a polynucleotide encoding a heterologous protein of interest will be operably linked to an appropriate expression control sequence for the particular host cell in which the heterologous protein is to be expressed. Any of a large number of well-known promoters can be used in such method. The choice of the promoter will depend on the expression levels to be achieved and on the desired cellular specificity. Additional elements such as polyadenylation signals, 5′ and 3′ untranslated sequences, etc. are also described in well-known reference books.

In metazoan (animals having the body composed of cells differentiated into tissues and organs) cells, promoters and other elements for expressing heterologous proteins are commonly used and are well known to those of skill. See, e. g., Cruz & Patterson (1973) Tissue Culture, Academic Press; Meth. Enzymology 68 (1979), Academic Press; Freshney, 3rd Edition (1994) Culture of Animal Cells: A Manual of Basic Techniques, Wiley-Liss. Promoters and control sequences for such cells include, e. g., the commonly used early and late promoters from Simian Virus 40 (SV40), or other viral promoters such as those from polyoma, adenovirus 2, bovine papilloma virus, or avian sarcoma viruses, herpes virus family (e. g., cytomegalovirus, herpes simplex virus, or Epstein-Barr Virus), or immunoglobulin promoters and heat shock promoters (see, e. g. Sambrook, Ausubel, Meth. Enzymology Pouwells, et al., supra (1987)). In addition, regulated promoters, such as metallothionein, (i. e., MT-1 and MT-2), glucocorticoid, or antibiotic gene “switches” can be used. Enhancer regions of such promoters can also be used.

Expression cassettes are typically introduced into a vector that facilitates entry of the expression cassette into a host cell and maintenance of the expression cassette in the host cell. Such vectors are commonly used and are well known to those of skill in the art. Numerous such vectors are commercially available, e. g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e. g., LEU2, URA3, TRP 1, HIS3, GFP), centromeric sequences, etc.

For expression in mammalian cells, any of a number of vectors can be used, such as pSV2, pBC12BI, and p91023, as well as lytic virus vectors (e. g., vaccinia virus, adenovirus, baculovirus), episomal virus vectors (e. g., bovine papillomavirus), and retroviral vectors (e. g., murine retroviruses).

As used herein, the term “disorder” refers to an ailment, disease, illness, clinical condition, or pathological condition.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment of an agent, e.g. identified using a method of screening of the invention, that is relatively non-toxic to the subject.

The term “therapeutic agent” refers to any molecule, compound, or treatment, that assists in the prevention or treatment of disorders, or complications of disorders.

Compositions comprising such an agent formulated in a compatible pharmaceutical carrier may be prepared, packaged, and labeled for treatment.

If the complex is water-soluble, then it may be formulated in an appropriate buffer, for example, phosphate buffered saline or other physiologically compatible solutions.

Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, rectal administration or, in the case of tumors, directly injected into a solid tumor.

The compounds may be formulated for parenteral administration by injection, e. g., by bolus injection or continuous infusion. Formulations for injection may be presented in unit dosage form, e. g., in ampoules or in multi-dose containers, with an added preservative.

The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e. g., sterile pyrogen-free water, before use.

The compounds may also be formulated as a topical application, such as a cream or lotion.

In addition to the formulations described previously, the compounds may also be formulated as a depot preparation. Such long acting formulations may be administered by implantation (for example, subcutaneously or intramuscularly) or by intramuscular injection.

Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the compositions in pharmaceutically acceptable form.

The composition in a vial of a kit may be in the form of a pharmaceutically acceptable solution, e. g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e. g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of compositions by a clinician or by the patient.

The lateral geniculate nucleus (LGN) (also called the lateral geniculate body or lateral geniculate complex) is a relay center in the thalamus for the visual pathway. It receives a major sensory input from the retina. The LGN is the main central connection for the optic nerve to the occipital lobe. In humans, each LGN has six layers of neurons (grey matter) alternating with optic fibers (white matter). The LGN is small, ovoid, ventral projection at the termination of optic tract on each side of the brain. The LGN and the medial geniculate nucleus which deals with auditory information are both thalamic nuclei and so are present in both hemispheres. The LGN receives information directly from the ascending retinal ganglion cells via the optic tract and from the reticular activating system. Neurons of the LGN send their axons through the optic radiation, a direct pathway to the primary visual cortex. In addition, the LGN receives many strong feedback connections from the primary visual cortex. In humans as well as other mammals, the two strongest pathways linking the eye to the brain are those projecting to the dorsal part of the LGN in the thalamus, and to the superior colliculus.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Examples

To address the question of whether LGN cell stimulation could evoke meaningful responses in blind or normal-sighted animal, the inventors infected either normal or blind mice with channelrhodopsin in the LGN via an AAV as well as with GCAMP in the cortex. This allowed them to stimulate the axon terminals of LGN cell in the cortex and observe if responses could be evoked in cortex. In their experiments the inventors found that channelrhodopsin expressed in the LGN cell axon terminals of mice could activate cells in acute slices of the visual cortex, where the LGN axon terminals were cut off from their cell bodies. They also found that V1 cells in vivo responded to such stimulation as well. These findings show that optogenetic stimulation of LGN axons is sufficient to evoke responses in visual cortex.

### Methods:

AAV Injection into the LGN and AAV Injection into V1:

Animals were anesthetized with FMM (fentanyl 0.05 mg/kg, medetomidine 0.5 mg/kg, midazolam 5.0 mg/kg). A 0.4 mm diameter hole was drilled on the left hemisphere of the skull at 2.25 mm posterior and 2.15 mm lateral from the bregma. A pipette filled with an adeno-associated virus (AAV) expressing Channelrhodopsin 2 (CatCh) fused to mcherry was lowered into the tissue. At 2.75 mm depth, the AAV was then injected into the tissue and the needle retracted. Injection of V1 with AAVs expressing GCAMP3 was at 2 and 3 mm lateral from the lambda to the left hemisphere of the brain and did not require activity guidance to target.

### Acute Slice Recording:

2 weeks after viral injection, the mouse brain was sliced at 150 microm thickness, cut in the sagittal plane in Cutting Solution (110 mM Choline chloride, 1.25 mM Sodium Phosphate Monobasic Dihydrate, 11.6 mM Sodium ascorbate, 25 mM D-Glucose, 2.5 mM Potassium Chloride, 7 mM Magnesium sulfate, 25 mM Sodium Bicarbonate, 0.5 mM Calcium chloride. After adding sodium bicarbonate, bubble with carbox for 30 min, then pH to 7.2-7.3 with 1M HCl. Add CaCl₂) at the final step. Osmolality should be 315 mM/kg.), allowed to rest in the cutting solution for 20 minutes, then moved to a heated chamber within a custom-built two-photon microscope, the chamber was filled with Mouse Ringer solution (10× Mouse Ringers: 1.1 M NaCl, 25 mM KCl, 10 mM CaCl₂), 16 mM MgCl2, 100 mM D-glucose. Before use, 900 mL of deionized water was bubbled with CO2 gas for 5 min and add 1.84 g of sodium bicarbonate (Sigma, S8875) and mix completely. Then add 100 mL of 10× Mouse Ringers. Next we warm it at 37° C. and bubble with 95% 02 and 5% CO2.) that was constantly being replaced with a vacuum filtration system. The two photon microscope recorded the GCAMP3 signals from the objective while blue light (emitted by a Thorlabs LED, cat. number M405LP1c) was projected onto the sample through a condenser below the slice.

### In Vivo Recording:

Mouse was anesthetized as when injected with AAV in the LGN and a cranial window over the primary visual cortex was made. The mouse was then placed onto a custom-build head holder under the 2-photon microscope. The GCAMP3 fluorescence was detected by the microscope while blue light was delivered to the tissue via a fiber optic placed adjacent to the objective.

1. A method for the treatment of blindness in a patient, wherein a vector comprising a gene coding for a light-sensitive molecule is injected into the lateral geniculate nucleus of the patient.

2. The method of claim 1 further comprising the step of exposing the visual cortex of said patient to light signals.

3. The method of claim 1 or 2 wherein the light-sensitive molecule is a halorhodopsin or a channelrhodopsin.

4. The method of claim 1, 2 or 3 wherein different vectors are injected at different location of the lateral geniculate nucleus of the patient.

5. The method of claim 4 wherein two different vectors are used, the first vector comprising a gene coding for a first light-sensitive molecule and the second vector comprising a gene coding for a second light-sensitive molecule.

6. The method of claim 5 wherein additional vectors are used, each of said additional vector comprising a gene coding for a different light-sensitive molecule.

7. The method of claim 4 or 5 wherein one of the light-sensitive molecules is a channelrhodopsin and another one is a halorhodopsin.

8. The method of any of claims 1 to 7 wherein the expression of the gene coding for a light-sensitive gene is under the control of a promoter selected from the group of Human elongation factor-1 alpha (EF-1 alpha), Human cytomegalovirus promoter (CMV) or CAG promoter.

9. A vector comprising a gene coding for a light-sensitive molecule for use in a method of treating blindness according to any of claims 1-8.

10. A vector according to claim 9, wherein the light-sensitive molecule is a halorhodopsin or a channelrhodopsin.

11. A vector according to claim 9 or 10 wherein the expression of the gene coding for a light-sensitive gene is under the control of a promoter selected from the group of Human elongation factor-1 alpha (EF-1 alpha), Human cytomegalovirus promoter (CMV) or CAG promoter.

12. A kit comprising at least two different vectors according to any of claims 9-11 for use in a method of treating blindness according to any of claims 1-8.
